(19) United States
(12) Patent Application Publication
(10) Pub. No.: US 2017/0356048 A1
(43) Pub. Date: Dec. 14, 2017

(54) METHOD FOR PREDICTING RECEPTIVITY TO TARGETED ANTICANCER DRUG

(71) Applicant: CBSBIOSCIENCE INC., Daejeon (KR)

(72) Inventors: Jin Young PARK, Seoul (KR); Young Ho Moon, Daejeon (KR); Jung-Hee Kwon, Daejeon (KR)

(21) Appl. No.: 15/328,432

(22) PCT Filed: Jul. 17, 2015
(86) PCT No.: PCT/KR2015/007425

§ 371 Date: Jan. 23, 2017

(30) Foreign Application Priority Data
Jul. 23, 2014 (KR) 10-2014-0093137

(51) Int. Cl. C12Q 1/68

(52) U.S. Cl. CPC C12Q 1/6886; C12Q 2600/106; C12Q 2600/158

(57) ABSTRACT

The present invention relates to a method for predicting sensitivity to treatment with a vascular endothelial growth factor receptor (VEGFR) inhibitor. According to the present invention, effects on the treatment of liver cancer patients with a vascular endothelial growth factor receptor (VEGFR) inhibitor are predicted. Based on the prediction, an effective means and anticancer therapy for treatment of liver cancer are selected, thereby increasing treatment effects and minimizing the side effects of liver cancer treatment.

### TECHNICAL FIELD

The present invention relates to a method of predicting the sensitivity of a liver cancer patient to treatment with a targeted anticancer drug to determine whether or not the patient can receive a therapeutic effect from the treatment.

### BACKGROUND

Cancer is one of the most fatal diseases that pose a threat to human health. In the USA, cancer affects about 1.3 million new patients every year, and is the second leading cause of death next to cardiovascular diseases, and accounts for about one-fourth of deaths. Although there has been a considerable advance in medical treatment of some cancers, the total five-year survival rate of all cancers has increased only to about 10% for the past 20 years. In addition, cancers or malignant tumors metastasize and abnormally and rapidly grow, and thus are very difficult to detect and treat in a timely manner.

Cancers are treated by various therapeutic methods, including chemotherapy, radiotherapy and antibody-based therapies, depending on the kind thereof, but the therapeutic effects of these therapeutic methods differ depending on the characteristics of patients and cancers.

Particularly, liver cancer is a carcinoma having high resistance to anticancer therapy, and many kinds of molecular targeted drugs have been studied for use as chemotherapeutic agents, but only some of these drugs have been approved by the FDA or the like and marketed. In addition, even anticancer drugs against liver cancer, which have been approved and marketed, show very low therapeutic effects depending on the nature of cancer in cancer patients and the lesion of cancer. For example, Nexavar (Sorafenib), a new drug approved for the purpose of treating hepatocellular carcinoma, shows a low treatment rate of about 2-3%, and the side effects thereof are also very high. Namely, current guidelines for treatment of hepatocellular carcinoma are not based on the molecular characteristics of tumors, and thus there is a limitation in that the therapeutic effects of the guidelines cannot be guaranteed.

Accordingly, there is a need for a more effective means for predicting whether an individual patient will respond to any treatment, and there is also a need for an effective anticancer therapeutic method in which the predicted outcome of an anticancer therapy selected using the means is applied to the patient.

### SUMMARY

The present invention is intended to provide a method for predicting sensitivity to a vascular endothelial growth factor receptor (VEGFR) inhibitor, the method comprising the steps of: measuring the expression levels of transcription factor 3 (TCF3) in samples isolated from liver cancer patients; and comparing the expression levels of the transcription factor 3 (TCF3) with a reference level, and selecting samples having a transcription factor 3 (TCF3) expression level equal to or higher than the reference level.

The present invention is also intended to provide a method for predicting sensitivity to a vascular endothclial growth factor receptor (VEGFR) inhibitor, the method comprising the steps of: measuring the expression levels of transcription factor 3 (TCF3) in samples isolated from liver cancer patients, and measuring the expression levels of any one or more selected from among cadherin 1 (CDH1), inhibitor of DNA binding 2 (ID2), and matrix metallopeptidase 9 (MMP9) in the samples; calculating the risk scores of the genes; and comparing the calculated risk scores with a reference level, and selecting samples having a risk score equal to or higher than the reference level.

The present invention is also intended to provide a composition for predicting sensitivity to a vascular endothelial growth factor receptor (VEGFR) inhibitor, the composition comprising an agent for measuring the mRNA expression level of any one or more selected from among transcription factor 3 (TCF3), cadherin 1 (CDH1), inhibitor of DNA binding 2 (ID2), and matrix metallopeptidase 9 (MMP9).

The present invention is also intended to provide a kit for predicting sensitivity to a vascular endothelial growth factor receptor (VEGFR) inhibitor, the kit comprising the composition.

The present invention is intended to provide a method for treating liver cancer using a vascular endothelial growth factor receptor (VEGFR) inhibitor, the method comprising the steps of: (a) measuring the expression levels of transcription factor 3 (TCF3) in samples isolated from liver cancer patients, and measuring the expression levels of any one or more selected from among cadherin 1 (CDH1), inhibitor of DNA binding 2 (ID), and matrix metallopeptidase 9 (MMP9) in the samples; (b) calculating the risk scores of the genes in step (a); (c) comparing the calculated risk scores with a reference level, and selecting samples having a risk score equal to or higher than the reference level; and (d) administering a therapeutically effective amount of the vascular endothelial growth factor receptor (VEGFR) inhibitor to a liver cancer patient having the risk score equal to or higher than the reference level.

The present invention is also intended to provide the use of an agent, which measures the mRNA expression level of any one or more selected from among transcription factor 3 (TCF3), cadherin 1 (CDH1), inhibitor of DNA binding 2 (ID2), and matrix metallopeptidase 9 (MMP9), for prediction of sensitivity to a vascular endothelial growth factor receptor (VEGFR) inhibitor.

### DETAILED DESCRIPTION

The present inventors have made extensive efforts to maximize the anticancer therapeutic effect by selecting patients having high sensitivity to the vascular endothelial growth factor receptor (VEGFR) inhibitor, and as a result, have found that sensitivity to the vascular endothelial growth factor receptor (VEGFR) inhibitor can be predicted according to the risk score calculated based on the expression levels of four markers (CDH1, ID2, MMP9 and TCF3), thereby completing the present invention.

The present invention provides a method for predicting sensitivity to a vascular endothelial growth factor receptor (VEGFR) inhibitor, the method comprising the steps of: measuring the expression levels of transcription factor 3 (TCF3) in samples isolated from liver cancer patients; and comparing the expression levels of the transcription factor 3 (TCF3) with a reference level, and selecting samples having a transcription factor 3 (TCF3) expression level equal to or higher than the reference level.

In the present invention, the vascular endothelial growth factor receptor (VEGFR) inhibitor is a therapeutic agent that exhibits an anticancer effect by inhibition of vascular endothelial growth factor receptor (VEGFR). For example, it may be anticancer drug such as brivanib, sunitinib, linifanib, regorafenib, sorafenib or the like. According to one embodiment of the present invention, the vascular endothelial growth factor receptor (VEGFR) inhibitor is sorafenib.

As used herein, the term “sensitivity” refers to the property capable of benefiting or providing a therapeutic effect from treatment with a vascular endothelial growth factor receptor (VEGFR) inhibitor that is a targeted anticancer drug. For example, the term means that the size of tumor is reduced by 5%, 10%, 15%, 20%, 25%, 30% or more after anticancer treatment. Preferably, the term means that the size of tumor is reduced by 30% or more (complete response and partial response rates according to Response Evaluation Criteria in Solid Tumors (RECIST), which are criteria for evaluating the responses of solid tumors to anticancer drugs).

Namely, according to the present invention, sensitivity to a vascular endothelial growth factor receptor (VEGFR) inhibitor is predicted, thereby predicting the possibility of treatment with the vascular endothelial growth factor receptor (VEGFR) inhibitor and the prognosis of the treatment and selecting an effective means and anticancer therapy for anticancer treatment to thereby increase therapeutic effects and minimize the side effects of cancer treatment.

The method for predicting sensitivity to the vascular endothelial growth factor receptor (VEGFR) inhibitor according to the present invention comprises a step of measuring the expression levels of TCF3 in samples isolated from liver cancer patients.

As used herein, the term “liver cancer patients” refers to patients having primary liver cancer that is a malignant tumor occurring in the liver. Preferably, the term refers to patients having hepatocellular carcinoma among hepatocellular carcinoma and intrahepatic bile duct cancer, which are primary liver cancers. In addition, the samples include whole blood, plasma, serum, tissue or the like, which are isolated from liver cancer patients. According to one embodiment of the present invention, liver tumor tissues are used. These samples may be suitably processed according to methods known in the art in order to measure expression levels.

In the present invention, measuring the expression levels includes measurement of the mRNA expression levels. As used herein, the expression “measuring the mRNA expression levels” refers to a process of measuring mRNA presence and expression level of a gene in a sample in order to predict sensitivity to a vascular endothelial growth factor receptor (VEGFR) inhibitor, and can be performed by measuring the level of mRNA. Analytical methods for measuring the mRNA expression levels include, but are not limited to, RT-PCR, competitive RT-PCR, real-time RT-PCR, RNase protection assay (RPA), Northern blotting, DNA chip assay and the like.

According to one embodiment of the present invention, mRNA expression levels are measured by quantitative real-time RT-PCR mRNA expression level can be quantified as 2^{-ΔCt} by the CT (the number of cycles required to reach a threshold value) of each gene and calculating the ΔCT value (the CT of each marker−the mean CT of reference genes). In the present invention, the expression level of transcription factor 3 (TCF3) is measured for the prediction of sensitivity to a vascular endothelial growth factor receptor (VEGFR) inhibitor. Transcription factor 3 (TCF3) is the transcription factor gene that regulates the development of B lymphocytes and T lymphocytes and the development of embryos, and has the nucleotide sequence of SEQ ID NO: 1.

The method for predicting sensitivity to the vascular endothelial growth factor receptor (VEGFR) inhibitor according to the present invention comprises a step of comparing the expression levels of TCF3 with a reference level and selecting samples having a TCF3 expression level equal to or higher than the reference level. When the expression level of TCF3 and the five-year prognosis (on the basis of death) of patients are assessed by univariate Cox regression analysis, the regression coefficient (RC) value for TCF3 has a positive value, because TCF3 positively acts as the expression level of TCF3 increases. For this reason, a sample having a TCF3 expression level equal to or higher than the reference level is selected. According to one embodiment of the present invention, regression coefficient is calculated from the five-year prognosis (on the basis of death) of 580 patients according to the same method as described in Cancer Science Vol. 101, No. 6, pp. 1521-1528 (2010).

As used herein, the term “reference level” refers to the same level as the expression level of genes capable of showing high diagnostic predictability, which is detected and identified from reference samples by the method described herein. A reference value can be relative to a number or value derived from population studies, including subjects having the same cancer, subjects having the same or similar age range, subjects in the same or similar ethnic group, subjects having family histories of cancer, or can be relative to the starting sample of a subject undergoing treatment for a cancer. Such reference values can be derived from statistical analyses and/or risk prediction data of populations obtained from mathematical algorithms and computed indices. Reference indices can also be constructed and used using algorithms and other methods of statistical and structural classification.

In certain embodiments, the term “reference level” herein refers to a predetermined value. For example, those skilled in the art will appreciate that the reference level is predetermined and set to meet routine requirements in terms of specificity, sensitivity and/or accuracy. For example, sensitivity or specificity, respectively, may be set to certain limits, e.g. 80%, 90% or 95%, respectively. These requirements may also be defined in terms of positive or negative predictive values. The reference level may be predetermined in healthy individuals or predetermined in the disease entity to which the patient belongs.

In the present invention, a statistical analysis method such as mean value calculation or ROC curve analysis may be used to determine the reference level of expression.

ROC curve analysis according to one embodiment of the present invention is performed in terms of sensitivity, specificity and accuracy using a curve that shows the performance of diagnosis. In the present invention, sensitivity is the ratio of true positives to the sum of persons having sensitivity to a vascular endothelial growth factor receptor (VEGFR) inhibitor, and specificity is the ratio of true negatives to the sum of persons having no sensitivity to a vascular endothelial growth factor receptor (VEGFR) inhibitor, and accuracy is the ratio of hits to all cases. When both specificity and sensitivity are high, the accuracy of test results increases. Thus, the x-axis in the ROC curve is 1-specificity, and the y-axis is sensitivity, and the AUC (area under curve) indicating accuracy means the area under the curve.

According to one embodiment of the present invention, “reference level” is a threshold value that shows a high predictive effect on the prediction of sensitivity to a vascular endothelial growth factor receptor (VEGFR) inhibitor. For the prediction of sensitivity, the expression level of transcription factor 3 (TCF3), which corresponds to an AUC value higher than the average level (about 0.6), preferably the highest AUC value, in the ROC curve analysis of TCF3, is selected as the best threshold value (criterion), and a sample having a TCF3 expression level equal to or higher than the reference level is predicted to have high sensitivity to a vascular endothelial growth factor receptor (VEGFR) inhibitor. Herein, the reference level for the gene expression level may be 0.273, preferably 0.3711.

As used herein, the term “more than” or “higher than” means a level higher than the reference level or means an overall increase of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample. As used herein, the term “less than” or “lower than” means a level lower than the reference level or means an overall decrease of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample.

The present invention also provides a method for predicting sensitivity to a vascular endothelial growth factor receptor (VEGFR) inhibitor, the method comprising the steps of: measuring the expression levels of transcription factor 3 (TCF3) in samples isolated from liver cancer patients, and measuring the expression levels of any one or more selected from among cadherin 1 (CDH1), inhibitor of DNA binding 2 (ID2), and matrix metallopeptidase 9 (MMP9) in the samples; calculating the risk scores of the genes; and comparing the calculated risk scores with a reference level, and selecting samples having a risk score equal to or higher than the reference level.

The method for predicting sensitivity to the vascular endothelial growth factor receptor (VEGFR) inhibitor according to the present invention may comprise, in addition to measurement of the expression level of transcription factor 3 (TCF3), measurement of the expression level of any one or more selected from among cadherin 1 (CDH1), inhibitor of DNA binding 2 (ID2), and matrix metallopeptidase 9 (MMP9).

In the present invention, cadherin 1 (CDH1) is the gene encoding cadherin 1 protein that mediates calcium-dependent intercellular adhesion, and it has the nucleotide sequence of SEQ ID NO: 2.

In the present invention, inhibitor of DNA binding 2 (ID2) is a gene that regulates cellular differentiation by inhibiting a transcription factor having a basic helix-loop-helix domain, and has the nucleotide sequence of SEQ ID NO: 3.

In the present invention, matrix metallopeptidase 9 (MMP9) is the gene encoding MMP9 enzyme belonging to the matrix metallopmteinase (MMP) family. It is involved in embryonic development, reproduction, cancer metastasis, and tissue remodeling, and has the nucleotide sequence of SEQ ID NO: 4.

Herein, the expression level of each of the genes, determined based on the results of Cox regression analysis, is as follows. The expression level of cadherin 1 (CDH1) is equal to or lower than a reference level for cadherin 1 (CDH1); the expression level of inhibitor of DNA binding 2 (ID2) is equal to or lower than a reference level for inhibitor of DNA binding 2 (ID2); and the expression level of matrix metallopeptidase 9 (MMP9) is equal to or higher than a reference level for matrix metallopeptidase 9 (MMP9).

In the present invention, the risk score of a combination of any one or more of the three genes with TCF3 is calculated to predict sensitivity to the vascular endothelial growth factor receptor (VEGFR) inhibitor. Namely, the method of the present invention may further comprise a step of calculating the risk score of the transcription factor 3 (TCF3), cadherin 1 (CDH1), inhibitor of DNA binding 2 (ID2), and matrix metallopeptidase 9 (MMP9).

The risk score can be generalized as the following equation 1 based on mRNA expression levels:

Equation 1

RISK(n)=(TCF3RC×TCF3 expression level)+(gene RC×gene expression level) \textcircled{1}

\textcircled{2} Risk score conversion: RISK(n) result values for 580 samples are obtained according to equation \textcircled{1}, and the result values are ranked from 1 to 580 and converted into risk scores between 0 and 100 (for example, ranked 1: 0.17 (1/580*100), ranked 580: 100 (580/580*100).

In the above equation, (n) is an integer ranging from 2 to 4, and the gene is any one or more of the three genes, and may comprise any one of the genes, or comprise two of the three genes, or comprise all the three genes. The “reference level” for the risk score is 60 or higher, for example, 60, 65, 70, 80, 85 or higher, and samples having a risk score equal to or higher than the reference level are selected and predicted to have high sensitivity to the vascular endothelial growth factor receptor (VEGFR) inhibitor.

According to one embodiment of the present invention, the mRNA expression levels of TCF3, CDH1, ID2 and MMP9 in samples are measured, and the risk score is calculated based on the measured expression levels. Samples having a risk score equal to or higher than the reference level are selected, thereby predicting sensitivity to a vascular endothelial growth factor receptor (VEGFR) inhibitor.

The risk score can be calculated using the following equation 2:

Equation 2

RISK4=(CDH1RC×CDH1 expression level)+(ID2RC×ID2 expression level)+(MMP9RC×MMP9 expression level)+(TCF3RC×TCF3 expression level) \textcircled{1}

\textcircled{2} Risk score conversion: RISK4 result values for 580 samples are obtained according to equation \textcircled{1}, and the result values are ranked from 1 to 580 and converted into risk scores between 0 and 100 (for example, ranked 1: 0.17 (1/580*100), ranked 580: 100 (580/580*100).

In equation \textcircled{1}, when the expression levels of the four genes and the five-year prognosis (death event) of the patients are assessed by Cox regression analysis, the regression coefficient (RC) value for CDH1 or ID2 has a negative value, because CDH1 or ID2 negatively acts as the expression level thereof increases. Furthermore, the regression coefficient (RC) value for TCF3 or MMP9 has a positive value, because TCF3 or MMP9 positively acts as the expression level thereof increases. The “reference level” is a threshold value showing a high predictive effect on the prediction of sensitivity to a vascular endothelial growth factor receptor (VEGFR) inhibitor, and the reference level for the risk score is 60 or higher, for example, 60, 65, 70, 80, 85 or higher. Samples having a risk score equal to or higher than the reference level are selected and predicted to have high sensitivity to the vascular endothelial growth factor receptor (VEGFR) inhibitor. Herein, the reference level for the risk score may be 60, preferably 87.59.

The present invention also provides a composition for predicting sensitivity to a vascular endothelial growth factor receptor (VEGFR) inhibitor, the composition comprising an agent for measuring the mRNA expression level of any one or more selected from among transcription factor 3 (TCF3), cadherin 1 (CDH1), inhibitor of DNA binding 2 (ID2), and matrix metallopeptidase 9 (MMP9).

The composition for predicting sensitivity to the vascular endothelial growth factor receptor (VEGFR) inhibitor makes it possible to predict sensitivity to the vascular endothelial growth factor receptor (VEGFR) inhibitor by measuring the mRNA expression level of any one or more of the four genes.

Preferably, the composition for predicting sensitivity to the vascular endothelial growth factor receptor (VEGFR) inhibitor may comprise any one or more of a primer pair, a probe and an anti-sense nucleotide, which are agents for measuring mRNA expression levels and bind specifically to the genes. The sequence of the above-described primer pair, probe or antisense nucleotide can be easily designed by those skilled in the art from the nucleotide sequences provided in the present invention. According to one embodiment of the present invention, the agent for measuring the mRNA expression level comprises a primer pair and a probe, and may have the primer pair and probe sequences shown in Table 1 below. According to other embodiments of the present invention, the composition of the present invention is a composition for predicting sensitivity to a vascular endothelial growth factor receptor (VEGFR) inhibitor, the composition comprising agents for measuring the mRNA expression levels of transcription factor 3 (TCF3), cadherin 1 (CDH1), inhibitor of DNA binding 2 (ID2), and matrix metallopeptidase 9 (MMP9).

The present invention also provides a kit for predicting sensitivity to a vascular endothelial growth factor receptor (VEGFR) inhibitor, the kit containing a composition for predicting sensitivity to the vascular endothelial growth factor receptor (VEGFR) inhibitor, the composition comprising an agent for measuring the mRNA expression level of any one or more selected from among transcription factor 3 (TCF3), cadherin 1 (CDH1), inhibitor of DNA binding 2 (ID2), and matrix metallopeptidase 9 (MMP9).

The kit makes it possible to predict sensitivity to the vascular endothelial growth factor receptor (VEGFR) inhibitor by measuring the expression level of any one or more of polynucleotides encoding the four proteins. The kit for predicting sensitivity to the vascular endothelial growth factor receptor (VEGFR) inhibitor according to the present invention may contain not only a primer pair or a probe for measuring expression levels, but also one or more other constituent compositions or devices suitable for analysis of the polynucleotides. Preferably, the diagnostic kit for quantitative detection of the above-described polynucleotides or genes according to the present invention may contain one or more oligonucleotides that bind specifically to the polynucleotides encoding the four proteins. Specifically, the diagnostic kit may contain primers corresponding to some sequences oligonucleotides, reverse transcriptase, Taq polymerase, primers for PCR, and dNTP. To measure polynucleotide expression levels, a kit employing an analytical method described with respect to “the measurement of mRNA expression levels” may be used. For example, the kit may be selected from among RT-PCR kits, competitive RT-PCR kits, real-time RT-PCR kits, real-time RT-PCR kits, and DNA chip kits.

According to some embodiments, the kit of the present invention contains a composition comprising agents for measuring the mRNA expression levels of transcription factor 3 (TCF3), cadherin 1 (CDH1), inhibitor of DNA binding 2 (ID2), and matrix metallopeptidase 9 (MMP9).

The present invention also provides a method for treating liver cancer using a vascular endothelial growth factor receptor (VEGFR) inhibitor, the method comprising the steps of: (a) measuring the expression levels of transcription factor 3 (TCF3) in samples isolated from liver cancer patients, and measuring the expression levels of any one or more selected from among cadherin 1 (CDH1), inhibitor of DNA binding 2 (ID2), and matrix metallopeptidase 9 (MMP9) in the samples; (b) calculating the risk scores of the genes in step (a); (c) comparing the calculated risk scores with a reference level, and selecting samples having a risk score equal to or higher than the reference level; and (d) administering a therapeutically effective amount of the vascular endothelial growth factor receptor (VEGFR) inhibitor to a liver cancer patient having the risk score equal to or higher than the reference level.

The vascular endothelial growth factor receptor (VEGFR) inhibitor is any one or more selected from among brivanib, sunitinib, linifanib, regorafenib, and sorafenib. Furthermore, the method may further comprise a step of administering a therapeutically effective amount of a therapeutic agent selected from the group consisting of cytotoxic agents, chemotherapeutic agents, growth inhibitors, antiangiogenic agents, and combinations thereof.

The present invention also provides the use of an agent, which measures the mRNA expression level of any one or more selected from among transcription factor 3 (TCF3), cadherin 1 (CDH1), inhibitor of DNA binding 2 (ID2), and matrix metallopeptidase 9 (MMP9), for prediction of sensitivity to a vascular endothelial growth factor receptor (VEGFR) inhibitor.

According to the present invention, effects on the treatment of liver cancer patients with a vascular endothelial growth factor receptor (VEGFR) inhibitor are predicted. Based on the prediction, an effective means and anticancer therapy for treatment of liver cancer are selected, thereby increasing treatment effects and minimizing the side effects of liver cancer treatment.

### DESCRIPTION OF DRAWINGS

FIG. 1 shows the results of analyzing the risk score distribution of responders and non-responders to sorafenib by use of CDH1, ID2, MMP9 and TCF3 genes.

FIG. 2 shows the results of analyzing the risk score distribution of responders to sorafenib and non-responders to sorafenib by use of each of CDH1, ID2, MMP9 and TCF3 genes.

FIG. 3 shows the results of receiver operating characteristic (ROC) analysis and Fisher's exact test analysis performed to confirm the ability of risk scores to predict and classify responders to sorafenib and non-responders to sorafenib.

The advantages and features of the present invention, and the way of attaining them, will become apparent with reference to the examples described below. However, the present invention is not limited to the examples disclosed below and can be embodied in a variety of different forms; rather, these examples are provided so that this disclosure will be thorough and complete, and will fully convey the scope of the present invention to those skilled in the art. The scope of the present invention will be defined by the appended claims.

### Examples

### Example 1: RNA Extraction and cDNA Synthesis

From 29 liver cancer patients who were diagnosed as having liver cancer, received hepatectomy or liver transplantation, showed recurrence of liver cancer, and received treatment with Nexavar (Sorafenib) in Ajou University Medical Center, Korea University Anam Hospital and Keimyung University Dongsan Medical Center, tissues were obtained under informed consent. In the following manner, RNA was extracted from each of the tissues and synthesized into cDNA.

Total RNA was extracted from liver cancer tissue and the surrounding normal tissue using an RNeasy Mini kit (Qiagen, Germany) according to the manufacturer's guideline. The extracted total RNA was quantified using Bioanalyzer 2100 (Agilent Technologies, USA). In the extraction step, the RNA extract was treated with DNase I to remove genomic DNA. Each sample comprising 4 μg of total RNA was incubated with 2 μl of 1 μM oligo d(T)18 primer (Genotech, Korea) at 70° C. for 7 minutes and cooled on ice for 5 minutes. A total of 1 μl of an enzyme mixture [2 μl of 0.1 M DTT (Duchefa, the Netherlands), 2 μl of 10× reverse transcription buffer, 5 μl of 2 mM dNTP, 1 μl of 200 U/μl MMLV reverse transcriptase and 1 μl of 40 U/μl RNase inhibitor (Enzynomics, Korea)] was separately prepared. The enzyme mixture was added to the mixture containing the RNA, and was incubated at 42° C. for 90 minutes, and then incubated at 80° C. for 10 minutes to inactivate the reverse transcriptase. Diethyl pyrocarbonate (DEPC)-treated water was added to the mixture to a final volume of 400 μl.

### Example 2: Quantitative Real-Time PCR

In order to measure the mRNA expression levels of CDH1, ID2, MMP9 and TCF3 markers selected as markers, for each of the cDNA samples obtained in Example 1, the gene markers shown in Table 1 below were amplified by real-time PCR using PRISM 7900HT (Applied Biosystems, USA) according to the manufacturer's guideline.

Real-time PCR analysis was performed using a total of 10 μl of a volume consisting of 5 μl of 2× TaqMan gene expression master mix (Applied Biosystems, USA), 1 μl of each of 5 μM forward and reverse primers, 1 μl of 1 μM probe (Genotech, Korea) and 2 μl of cDNA (the same amount of water for a control group). The PCR amplification consisted of dissociation at 95° C. for 10 minutes, followed by cycles, each consisting of dissociation at 95° C. for 22 seconds and synthesis at 60° C. for 1 minute. The primer and probe sequences were designed using Primer Express 3.0 (Applied Biosystems, USA), and all the probe sequences were labeled with FAM at the 5′ end and TAMRA at the 3′ end. For each of the markers, the primer and probe sequences shown in Table 1 below were used.

TABLE 1
Information of primer and probe sequences for
marker genes

| Gene | | Sequence |
|---|---|---|
| CDH1 | F | AAATCTGAAAGCGGCTGATACTG (SEQ ID NO: 5) |
| | R | CGGAACCGCTTCCTTCATAG (SEQ ID NO: 6) |
| | P | CCCCACAGCCCCGCCTTATGA (SEQ ID NO: 7) |
| ID2 | F | AACGACTGCTACTCCAAGCTCAA (SEQ ID NO: 8) |
| | R | GGATTTCCATCTTGCTCACCTT (SEQ ID NO: 9) |
| | P | TGCCCAGCATCCCCCAGAACAA (SEQ ID NO: 10) |
| MMP9 | F | GGGCTCCCGTCCTGCTT (SEQ ID NO: 11) |
| | R | ACTCCTCCCTTTCCTCCAGAAC (SEQ ID NO: 12) |
| | P | TGCCATGTAAATCCCCACTGGGACC (SEQ ID NO: 13) |
| TCF3 | F | GCTGCCTTTGGTCTCTGGTTT (SEQ ID NO: 14) |
| | R | AGAAATGCAATGCTCAGTCTAGGA (SEQ ID NO: 15) |
| | P | AGTCCCGTGTCTCTCGCTATTTCTGCTG |
| | | (SEQ ID NO: 16) |

The expression of each marker gene was repeatedly measured three times, and normalized to the average expression of five reference genes (B2M, GAPDH, HMBS, IIPRT1, and SDHA). For the reference genes, the primer and probe sequences shown in Table 2 below were used.

TABLE 2
Information of primer and probe sequences
for reference genes

| Gene | | Sequence |
|---|---|---|
| B2M | F | CATTCGGGCCGAGATGTCT (SEQ ID NO: 17) |
| | R | CTCCAGGCCAGAAAGAGAGAGTAG (SEQ ID NO: 18) |
| | P | CCGTGGCCTTAGCTGTGCTCGC (SEQ ID NO: 19) |
| GAPDH | F | CACATGGCCTCCAAGGAGTAA (SEQ ID NO: 20) |
| | R | TGAGGGTCTCTCTCTTCCTCTTGT (SEQ ID NO: 21) |
| | P | CTGGACCACCAGCCCCAGCAAG (SEQ ID NO: 22) |
| HMBS | F | CCAGGGATTTGCCTCACCTT (SEQ ID NO: 23) |
| | R | AAAGAGATGAAGCCCCCACAT (SEQ ID NO: 24) |
| | P | CCTTGATGACTGCCTTGCCTCCTCAG |
| | | (SEQ ID NO: 25) |
| HPRT1 | F | GCTCGAGATGTGATGAAGGAGAT (SEQ ID NO: 26) |
| | R | CCAGCAGGTCAGCAAAGAATT (SEQ ID NO: 27) |
| | P | CCATCACATTGTAGCCCTCTGTGTGCTC |
| | | (SEQ ID NO: 28) |
| SDHA | F | CACCTAGTGGCTGGGAGCTT (SEQ ID NO: 29) |
| | R | GCCCAGTTTTATCATCTCACAAGA (SEQ ID NO: 30) |
| | P | TGGCACTTACCTTTGTCCCTTGCTTCA |
| | | (SEQ ID NO: 31) |

The CT (the number of cycles required to reach a threshold value) of each marker was measured, and the ΔCT value (the CT of each marker−the mean CT of reference genes) was calculated. The mRNA expression level of each marker was calculated as 2^{-ΔCt}.

### Example 3: Statistical Analysis

Using the expression levels (2^{-ΔCt}) of the four markers obtained in Example 2, a total of three statistical analyses (risk score, individual gene expression distribution analysis and ROC curve analysis) were performed in the following manner.

(1) Risk Score Calculation

In order to examine the correlation between the therapeutic effect of sorafenib known to have a therapeutic effect of about 2.3% [see (NEJM2008 359(4):378-390)] and risk scores based on the expression levels of the four markers, the risk scores were calculated.

For risk score calculation, the value (log 2 transformed 2^{-ΔCt}) obtained from the expression level of each marker in tumor tissue, obtained in Example 2, was multiplied by regression coefficient (RC), and then the total sum was calculated. For risk score calculation, the following equation 2 was used:

Equation 2

RISK4=(CDH1RC×CDH1 expression level)+(ID2RC×ID2 expression level)+(MMP9RC×MMP9 expression level)+(TCF3RC×TCF3 expression level) \textcircled{1}

\textcircled{2} Risk score conversion: RISK4 result values for 580 samples are obtained according to equation \textcircled{1}, and the result values are ranked from 1 to 580 and converted into risk scores between 0 and 100 (for example, ranked 1: 0.17 (1/580*100), ranked 580: 100 (580/580*100).

The regression coefficient (RC) of each gene used \textcircled{1} above was the value shown in Table 3 below. The regression coefficient was calculated based on death data of 580 persons according to the method described in Cancer Science Vol. 101, No. 6, pp. 1521-1528 (2010), and the calculated regression coefficient values are shown in Table 3 below.

TABLE 3
Regression coefficients

| Marker | Regression Coefficient |
|---|---|
| CDH1 | −0.124 |
| ID2 | −0.314 |
| MMP9 | 0.011 |
| TCF3 | 0.170 |

The risk scores of a total of 29 liver cancer patients were calculated, and the risk score distribution of responders to sorafenib and the risk score distribution of non-responders to sorafenib were examined. Herein, whether or not sorafenib would have a therapeutic effect was determined based on the degree of reduction in tumor size by CT and MRI imagings, and criteria for the determination followed mRECIST (modified Response Evaluation Criteria in Solid tumors) criteria version 1.1.

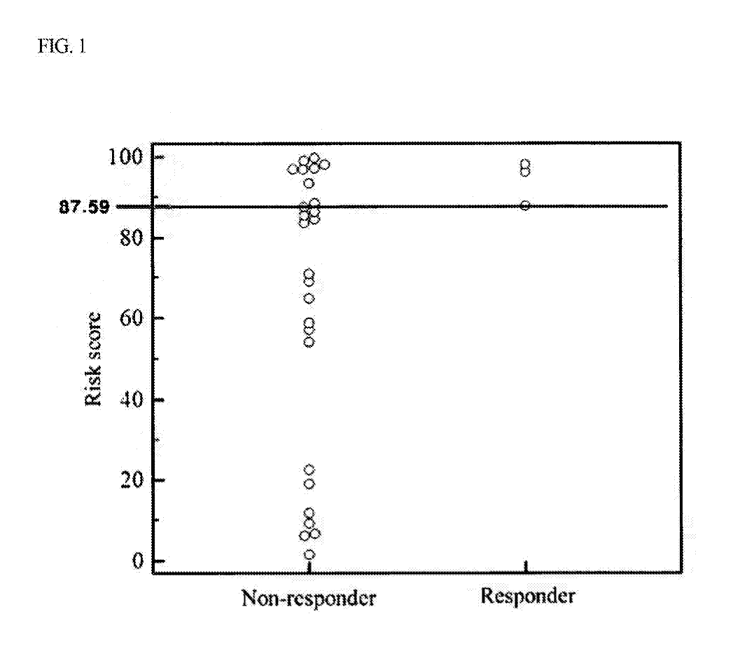

FIG. 1 shows the results of calculating the risk scores of responders to sorafenib and non-responders to sorafenib. As shown in FIG. 1, the responders had high risk scores compared to the non-responders, and this difference was statistically significant (the mean risk scores of the non-responders and the responders were 64.49 and 94.06, respectively; p=0.000523).

(2) Analysis of Expression Distribution of Individual Markers

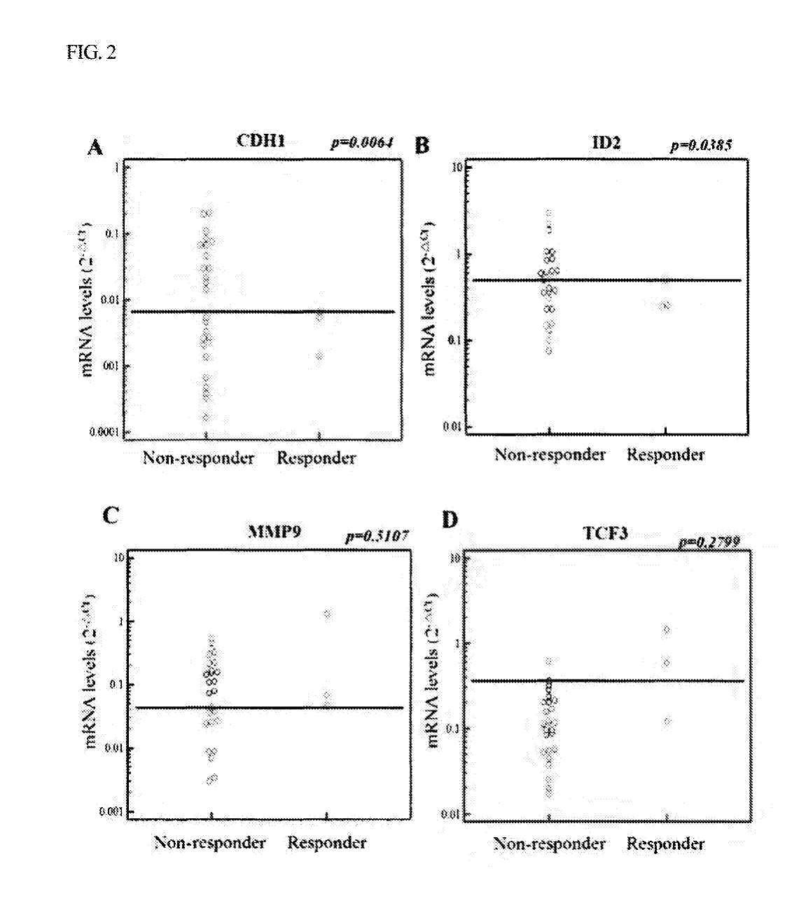

In order to examine the correlation between the effect of sorafenib and the expression levels (2^{-ΔCt}) of the four individual markers, which constitute the risk scores, the expression levels of the four individual markers and the distribution thereof were analyzed for responders to sorafenib and non-responders to sorafenib, and the results of the analysis are shown in FIG. 2.

FIG. 2 shows the results of analyzing the mRNA expression levels of four marker genes (CDH1, ID2, MMP9 and TCF3) for responders and non-responders.

When expression of each of these markers was examined, it was shown in FIG. 2A that the expression level of CDH1 was down-regulated in the responders (mean value of 2^{-ΔCt}: 0.005) compared to in the non-responders (mean value of 2^{-ΔCt}: 0.038), with statistical significance (0.0064). Furthermore, it was shown in FIG. 2B that the expression level of ID2 was down-regulated in the responders (mean value of 2^{-ΔCt}: 0.33163) compared to in the non-responders (mean value of 2^{-ΔCt}: 0.68765), with statistical significance (0.0385). Moreover, it was shown in FIG. 2C that the expression level of MMP9 was up-regulated in the responders (mean value of 2^{-ΔCt}: 0.4687) compared to in the non-responders (mean value of 2^{-ΔCt}: 0.1417). In addition, it was shown in FIG. 2D that the expression level of TCF3 was up-regulated in the responders (mean value of 2^{-ΔCt}: 0.7189) compared to in the non-responders (mean value of 2^{-ΔCt}: 0.15166).

(3) ROC Curve Analysis

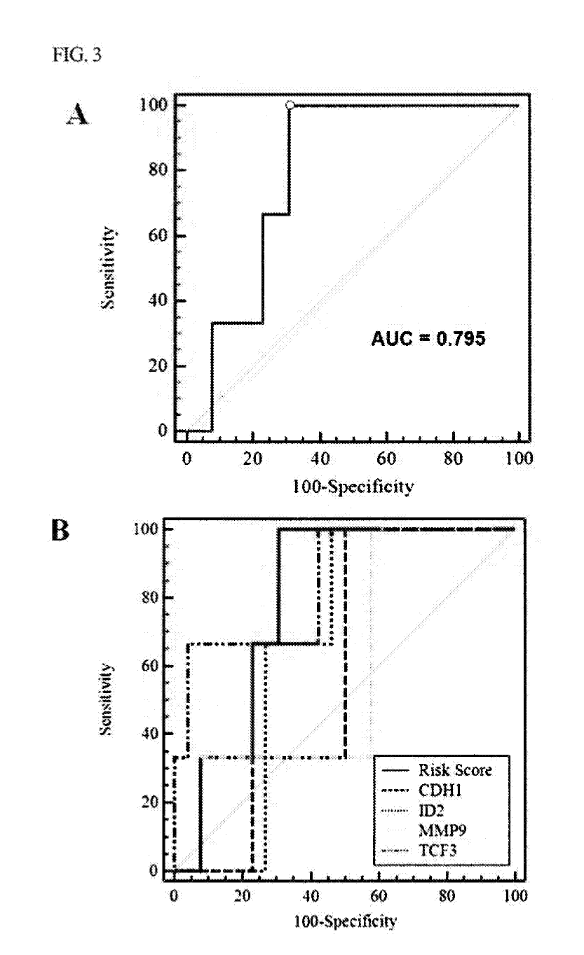

In order to confirm whether the risk scores of 29 patients, calculated as described above, have the ability to predict and classify responders to sorafenib and non-responders to sorafenib, ROC (receiver operating characteristic) curve analysis and Fisher's exact test analysis were performed using various cut-off values. Furthermore, based on the gene expression level corresponding to the average AUC value (about 0.6) or the highest AUC value in the ROC curve, the best threshold value (criterion) was obtained, and sensitivity, specificity, AUC value and p value, which correspond to each classifier and criterion, were obtained. The results of the analysis are shown in Tables 4 and 5 below and FIG. 3.

FIG. 3A shows the results of ROC curve analysis performed to predict a response to sorafenib by risk scores. The results of ROC curve analysis indicated that the highest AUC was 0.795 (p=0.0020).

FIG. 3B shows the results of ROC curve analysis performed to predict a response to sorafenib by the mRNA expression level of each marker gene. It was shown that the highest AUC values in ROC curve analysis were 0.590, 0.667, 0.615 and 0.846 for CDH1, ID2, MMP9 and TCF3, respectively.

The results of the analysis are summarized in Table 4 below, and it was shown that TCF3 among the individual markers showed the highest AUC values.

TABLE 4
Sensitivity, specificity and accuracy of the prediction of response
to sorafenib treatment by risk score and each marker gene

| | | Sensitivity | Specificity | | |
|---|---|---|---|---|---|
| Classifiers | Criterion | (%) | (%) | AUC | p value |
| Risk score | >60.0 | 100 | 41.0 | 0.6123 | 0.0124 |
| | >87.59 | 100 | 69.2 | 0.795 | 0.0020 |
| CDH1 | ≦0.128 | 100 | 38.4 | 0.6213 | 0.5124 |
| | ≦0.0067 | 100 | 50.0 | 0.590 | 0.4687 |
| ID2 | ≦0.2415 | 100 | 36.4 | 0.6174 | 0.2637 |
| | ≦0.4965 | 100 | 53.9 | 0.667 | 0.1167 |
| MMP9 | >0.3213 | 100 | 30.4 | 0.6328 | 0.6087 |
| | >0.0433 | 100 | 42.3 | 0.615 | 0.5703 |
| TCF3 | >0.2731 | 100 | 39.4 | 0.607 | 0.031 |
| | >0.3711 | 66.7 | 96.2 | 0.846 | 0.0136 |

In addition, after stratification of the liver cancer patients by classification, the rate of response to sorafenib treatment was examined in order to confirm improvement in clinical responses, and the results of the examination are shown in Table 5 below.

TABLE 5
Rate of response to sorafenib treatment in patients classified
based on risk score and gene expression level

| | | | | Response | | |
|---|---|---|---|---|---|---|
| | | | Non- | rate | P | odds |
| Classifiers | criterion | Responders | responders | (%) | value | ratio |
| Risk score | ≦87.59 | 0 | 18 | 27.27 | 0.04516 | 0 |
| | >87.59 | 3 | 8 | | | |
| CDH1 | ≦0.0067 | 3 | 13 | 18.75 | 0.2315 | Inf |
| | >0.0067 | 0 | 13 | | | |
| ID2 | ≦0.4965 | 3 | 12 | 20.0 | 0.2241 | Inf |
| | >0.4965 | 0 | 14 | | | |
| MMP9 | ≦0.0433 | 0 | 11 | 16.67 | 0.2685 | 0 |
| | >0.0433 | 3 | 15 | | | |
| TCF3 | ≦0.3711 | 1 | 25 | 66.67 | 0.02162 | 0.029764 |
| | >0.3711 | 2 | 1 | | | |

As can be seen in Table 5 above, 11 HCC patients screened based on the best threshold value corresponding to the highest AUC showed a risk score higher than 87.59 for the four markers, and showed a response rate of 27.27%, and there was a considerable correlation between the risk score and the response to the drug (p=0.0415). In addition, it was shown that, among the individual marker genes, TCF3 showed the highest response rate (66.67%) and that there was a considerable correlation between the risk score and the response to the drug (p=0.02162).

1. A method for predicting sensitivity to a vascular endothelial growth factor receptor (VEGFR) inhibitor, the method comprising the steps of:
(a) measuring expression levels of transcription factor 3 (TCF3) in samples isolated from liver cancer patients; and
(b) comparing the expression levels of the transcription factor 3 (TCF3) with a reference level, and selecting samples having a transcription factor 3 (TCF3) expression level equal to or higher than the reference level.

2. The method of claim 1, wherein the step (a) comprises additionally measuring expression levels of any one or more selected from among cadherin 1 (CDH1), inhibitor of DNA binding 2 (ID2), and matrix metallopeptidase 9 (MMP9) in the samples.

3. The method of claim 2, wherein the step (a) measures expression levels of the transcription factor 3 (TCF3), cadherin 1 (CDH1), inhibitor of DNA binding 2 (ID2), and matrix metallopeptidase 9 (MMP9) in the samples.

4. The method of claim 1, wherein the reference level is 0.273 as mRNA expression level of the transcription factor 3 (TCF3).

5. The method of claim 2, wherein the reference level is 60 or more for a risk score.

6. The method of claim 1, wherein the vascular endothelial growth factor receptor (VEGFR) inhibitor is any one or more selected from the group consisting of brivanib, sunitinib, linifanib, regorafenib, and sorafenib.

7. The method of claim 1, wherein the measuring of the expression levels in step (a) is measurement of mRNA expression levels.

8. The method of claim 1, wherein the transcription factor (TCF3) has a nucleotide sequence of SEQ ID NO: 1.

9. The method of claim 2, wherein the cadherin 1 (CDH1) has a nucleotide sequence of SEQ ID NO: 2, the inhibitor of DNA binding 2 (ID2) has a nucleotide sequence of SEQ ID NO: 3, and the matrix metallopeptidase 9 (MMP9) has a nucleotide sequence of SEQ ID NO: 4.

10. A method for predicting sensitivity to a vascular endothelial growth factor receptor (VEGFR) inhibitor comprising administering an agent for measuring mRNA expression levels of any one or more genes selected from among transcription factor 3 (TCF3), cadherin 1 (CDH1), inhibitor of DNA binding 2, and matrix metallopeptidase 9 (MMP9) to samples isolated from liver cancer patients.

11. The method of claim 10, wherein the agent measures the mRNA expression levels of transcription factor 3 (TCF3), cadherin 1 (CDH1), inhibitor of DNA binding 2, and matrix metallopeptidase 9 (MMP9).

12. The method of claim 10, wherein the vascular endothelial growth factor receptor (VEGFR) inhibitor is any one or more selected from the group consisting of brivanib, sunitinib, linifanib, regorafenib, and sorafenib.

13. The method of claim 10, wherein the agent for measuring the mRNA expression levels comprises primer pairs, probes or antisense nucleotides, which bind specifically to the genes.

14. The method of claim 13, wherein the agent for measuring the mRNA expression levels comprises primer pairs and probes shown in the following Table, which bind specifically bind to the genes:

| Gene | | Sequence |
|---|---|---|
| CDH1 | Forward primer | AAATCTGAAAGCGGCTGATACTG (SEQ ID NO: 5) |
| | Reverse primer | CGGAACCGCTTCCTTCATAG (SEQ ID NO: 6) |
| | Probe | CCCCACAGCCCCGCCTTATGA (SEQ ID NO: 7) |
| ID2 | Forward primer | AACGACTGCTACTCCAAGCTCAA (SEQ ID NO: 8) |
| | Reverse primer | GGATTTCCATCTTGCTCACCTT (SEQ ID NO: 9) |
| | Probe | TGCCCAGCATCCCCCAGAACAA (SEQ ID NO: 10) |
| MMP9 | Forward primer | GGGCTCCCGTCCTGCTT (SEQ ID NO: 11) |
| | Reverse primer | ACTCCTCCCTTTCCTCCAGAAC (SEQ ID NO: 12) |
| | Probe | TGCCATGTAAATCCCCACTGGGACC (SEQ ID NO: 13) |
| TCF3 | Forward primer | GCTGCCTTTGGTCTCTGGTTT (SEQ ID NO: 14) |
| | Reverse primer | AGAAATGCAATGCTCAGTCTAGGA (SEQ ID NO: 15) |
| | Probe | AGTCCCGTGTCTCTCGCTATTTCTGCTG (SEQ ID NO: 16) |

15.-16. (canceled)

17. A method for treating liver cancer, comprising the steps of:
(a) measuring expression levels of transcription factor 3 (TCF3) in samples isolated from liver cancer patients, and measuring expression levels of any one or more selected from among cadherin 1 (CDH1), inhibitor of DNA binding 2, and matrix metallopeptidase 9 (MMP9) in the samples;
(b) calculating risk scores of the genes in step (a);
(c) comparing the calculated risk scores with a reference level, and selecting samples having a risk score equal to or higher than the reference level; and
(d) administering a therapeutically effective amount of a vascular endothelial growth factor receptor (VEGFR) inhibitor to a liver cancer patient having a risk score equal to or higher than the reference level.

18. The method of claim 17, wherein the vascular endothelial growth factor receptor (VEGFR) inhibitor is any one or more selected from the group consisting of brivanib, sunitinib, linifanib, regorafenib, and sorafenib.

19. (canceled)


SEQUENCE LISTING (31 sequences)

SEQ ID NO: 1 - DNA, 4462 bp, Homo sapiens
  DNA (1)..(4462): transcription factor 3
gcgccgcgtgcccggccgcgcccagcagggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaacggccactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaatgtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagcccccccgtccccccccccccccggaagccactgcacacagacagaacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggcccccagggctaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactcctttgccccagggacccaggctcatgctctgaactctggctgagaggatgctgctcaggagccagcacaggacaccccccaccccaccccaccatgtccccattacaccagagggccatcgtgacgtagacaggatgccaggggcctggccagcctcccccaatgctggggagcatccctgggcctggggccacacctgctgccctccctctgtgtggtccaagggcaagagtggctggagccgggggactgtgctggtctgagccccacgaaggccttgggctgtgcgtccgaccctgctgcagaaccagcagggtgtcccctcgggcccatctgtgtcccatgtcccagcacccaggcctctctccaggtctccttttctggtcttttgccatgagggtaaccagctcttcccagctggctggggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactcccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctacgccctgggagtttgatctcttaagggaacttgcctctccctcttgttttgctcctggccctgcccctaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgaaaaaaaaaaaaaaaaa

SEQ ID NO: 2 - DNA, 4815 bp, Homo sapiens
  DNA (1)..(4815): cadherin 1
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 3 - DNA, 1402 bp, Homo sapiens
  DNA (1)..(1402): inhibitor of DNA binding 2
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 4 - DNA, 2387 bp, Homo sapiens
  DNA (1)..(2387): matrix metallopeptidase 9
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 5 - DNA, 23 bp, Artificial Sequence
  Forward primer of CDH1
  DNA (1)..(23)
aaatctgaaagcggctgatactg

SEQ ID NO: 6 - DNA, 20 bp, Artificial Sequence
  Reverse primer of CDH1
  DNA (1)..(20)
cggaaccgcttccttcatag

SEQ ID NO: 7 - DNA, 21 bp, Artificial Sequence
  CDH1 Probe
  DNA (1)..(21)
ccccacagccccgccttatga

SEQ ID NO: 8 - DNA, 23 bp, Artificial Sequence
  Forward primer of ID2
  DNA (1)..(23)
aacgactgctactccaagctcaa

SEQ ID NO: 9 - DNA, 22 bp, Artificial Sequence
  Reverse primer of ID2
  DNA (1)..(22)
ggatttccatcttgctcacctt

SEQ ID NO: 10 - DNA, 22 bp, Artificial Sequence
  ID2 Probe
  DNA (1)..(22)
tgcccagcatcccccagaacaa

SEQ ID NO: 11 - DNA, 17 bp, Artificial Sequence
  Forward primer of MMP9
  DNA (1)..(17)
gggctcccgtcctgctt

SEQ ID NO: 12 - DNA, 22 bp, Artificial Sequence
  Reverse primer of MMP9
  DNA (1)..(22)
actcctccctttcctccagaac

SEQ ID NO: 13 - DNA, 25 bp, Artificial Sequence
  MMP9 Probe
  DNA (1)..(25)
tgccatgtaaatccccactgggacc

SEQ ID NO: 14 - DNA, 21 bp, Artificial Sequence
  Forward primer of TCF3
  DNA (1)..(21)
gctgcctttggtctctggttt

SEQ ID NO: 15 - DNA, 24 bp, Artificial Sequence
  Reverse primer of TCF3
  DNA (1)..(24)
agaaatgcaatgctcagtctagga

SEQ ID NO: 16 - DNA, 28 bp, Artificial Sequence
  TCF3 Probe
  DNA (1)..(28)
agtcccgtgtctctcgctatttctgctg

SEQ ID NO: 17 - DNA, 19 bp, Artificial Sequence
  Forward primer of B2M
  DNA (1)..(19)
cattcgggccgagatgtct

SEQ ID NO: 18 - DNA, 24 bp, Artificial Sequence
  Reverse primer of B2M
  DNA (1)..(24)
ctccaggccagaaagagagagtag

SEQ ID NO: 19 - DNA, 22 bp, Artificial Sequence
  B2M probe
  DNA (1)..(22)
ccgtggccttagctgtgctcgc

SEQ ID NO: 20 - DNA, 21 bp, Artificial Sequence
  Forward primer of GAPDH
  DNA (1)..(21)
cacatggcctccaaggagtaa

SEQ ID NO: 21 - DNA, 24 bp, Artificial Sequence
  Reverse primer of GAPDH
  DNA (1)..(24)
tgagggtctctctcttcctcttgt

SEQ ID NO: 22 - DNA, 22 bp, Artificial Sequence
  GADPH Probe
  DNA (1)..(22)
ctggaccaccagccccagcaag

SEQ ID NO: 23 - DNA, 20 bp, Artificial Sequence
  Forward primer of HMBS
  DNA (1)..(20)
ccagggatttgcctcacctt

SEQ ID NO: 24 - DNA, 21 bp, Artificial Sequence
  Reverse primer of HMBS
  DNA (1)..(21)
aaagagatgaagcccccacat

SEQ ID NO: 25 - DNA, 26 bp, Artificial Sequence
  HMBS Probe
  DNA (1)..(26)
ccttgatgactgccttgcctcctcag

SEQ ID NO: 26 - DNA, 23 bp, Artificial Sequence
  Forward primer of HPRT1
  DNA (1)..(23)
gctcgagatgtgatgaaggagat

SEQ ID NO: 27 - DNA, 21 bp, Artificial Sequence
  Reverse primer of HPRT1
  DNA (1)..(21)
ccagcaggtcagcaaagaatt

SEQ ID NO: 28 - DNA, 28 bp, Artificial Sequence
  HPRT1 Probe
  DNA (1)..(28)
ccatcacattgtagccctctgtgtgctc

SEQ ID NO: 29 - DNA, 20 bp, Artificial Sequence
  Forward primer of SDHA
  DNA (1)..(20)
cacctagtggctgggagctt

SEQ ID NO: 30 - DNA, 24 bp, Artificial Sequence
  Reverse primer of SDHA
  DNA (1)..(24)
gcccagttttatcatctcacaaga

SEQ ID NO: 31 - DNA, 27 bp, Artificial Sequence
  SDHA Probe
  DNA (1)..(27)
tggcacttacctttgtcccttgcttca